(19) United States
(12) Patent Application Publication
(10) Pub. No.: US 2003/0174809 A1
(43) Pub. Date: Sep. 18, 2003

(54) Method and apparatus for the three-dimensional location of a concrement

(75) Inventor: Jurgen Neumann, Alling (DE)

Correspondence Address: GRAY CARY WARE & FREIDENRICH LLP, 2000 UNIVERSITY AVENUE, E. PALO ALTO, CA, 94303-2248, US

(73) Assignee: Dornier MedTech Systems GmbH

(21) Appl. No.: 10/309,304

(22) Filed: Dec. 2, 2002

(30) Foreign Application Priority Data
Dec. 10, 2001 (DE) 10160532.3

(51) Int. Cl. A61N 5/10

(52) U.S. Cl. 378/65

(57) ABSTRACT

A method for locating a concrement (3), for example a renal calculus or gallstone, in the body (1) of an organism for positioning a therapy device such as a lithotripsy instrument, whereby a substantially vertical projection image of the concrement is generated on an image window (6) by means of a radiation source (5), the body is displaced in the body plane relative to the radiation source (5), the projection d′ of the displacement distance is captured with a further projection image and the vertical z-position of the concrement is calculated as

$$z = {z}^{′} \cdot \frac{d}{{d}^{′}} ,$$

where z′ is the fixed distance between the radiation source (5) and the image window (6).

### TECHNICAL FIELD

The invention relates to the location of concrements such as gallstones or renal calculi in the bodies of organisms for the positioning of therapy systems, in particular therapy systems for shock wave therapy.

### STATE OF THE ART

In the last fifteen to twenty years shock wave therapy or lithotripsy has established itself as a non-invasive form of therapy for treating patients with gallstones or renal or vesical calculi. The principle of shock wave therapy is illustrated schematically in FIG. 3. A shock wave source generates acoustic waves by means of a transducer, which waves are transmitted to the body 1 of the patient to be treated by means of a coupling pad 11 filled with gel-like or aqueous material. The shock wave field 12 is focused at a focal point F. To be able to disintegrate the concrement (the stone 3) in an organ 13 into sufficiently small fragments by means of the shock waves it is necessary for the focal point F of the shock wave field 12 to coincide with the concrement 3. Precise location of the stone 3 for subsequent positioning of the shock wave source 10 is therefore required.

The concrement 3 is frequently located by means of X-radiation, whereby X-radiation striking the body vertically and transilluminating same (AP-projection) is combined with X-radiation striking the same body surface area obliquely and transilluminating same (co-projection). By means of the vertical X-radiation the horizontal position (X-Y plane) of the concrement can be directly determined. The X-ray source is then rotated through a defined angle, for example 30° to the vertical, i.e. to the original direction of projection (AP), whereby the vertical position of the concrement can be determined. Then—with obliquely incident X-radiation while retaining the horizontal position—the patient to be treated is moved in the vertical direction by means of a treatment table until the stone to be eliminated is in the correct vertical position (z-direction) at the focal point of the shock wave source. A locating device of this kind is described in DE 19513400 A1.

The above-described location method has, however, the disadvantage that the oblique positioning of the frequently large and heavy X-ray appliances is difficult and, with certain designs, impossible. For certain applications, e.g. urological applications, the radiation direction of the X-ray source on to the patient is fixed, generally AP.

It is the object of the present invention to overcome the disadvantages present in the state of the art and to propose a method and an apparatus for locating concrements by means of an imaging device, whereby irradiation of the body from different spatial directions is not required.

### SUMMARY OF THE INVENTION

This object is achieved by a method for the three-dimensional location of a concrement in the body of an organism for positioning a therapy device whereby a substantially vertical projection image of the concrement is generated by means of an imaging radiation source, the image coordinates of the concrement are determined, the body is moved horizontally relative to the radiation source and the displacement distance d is captured, the projection d′ of the displacement distance is captured and the vertical position of the concrement is determined from the values d, d′ obtained, and from the distance z′ of the radiation source from the image plane of the imaging device.

Known from DE 1029122 are an apparatus and a measurement method for determining the position and size of foreign bodies in transilluminated bodies such as patients, whereby the horizontal and vertical position of an object in the body is determined by means of two markings B1, B2 on the observation screen of the X-ray appliance. The mode of operation of the apparatus is based on the law of the relationship between the sides of similar triangles. The vertical position of a concrement is determined by relating together the dimensions of similar triangles.

Using the method according to the invention it is possible to determine the complete three-dimensional position of a concrement in the body solely by means of two vertical projection images obtained, for example, by means of X-rays. It is only necessary for the body to be moved horizontally—in the plane of the body—by a displacement distance d relative to the radiation source. Oblique positioning of the radiation source is not, therefore, required.

The relative motion between body and radiation source can be effected by means of a horizontally-displaceable treatment table or a horizontally-displaceable imaging device.

The vertical distance z of the concrement from the radiation source is preferably determined according to the invention as

$$z = {z}^{′} \cdot \frac{d}{{d}^{′}} .$$

The accuracy of positioning increases with the length of the displacement distance d. In determining the vertical position of the concrement, therefore, the available image field of the imaging device is preferably utilised to the maximum, for example, the diagonal of a rectangular image field or the diameter of a circular image field is used.

The imaging radiation used can preferably be X-radiation. Any other radiation which supplies a vertical projection image of the body is, however, also usable.

In addition, the invention proposes an apparatus for locating a concrement in the body according to claim 8.

The subsidiary claims describe further preferred embodiments of the invention.

### BRIEF DESCRIPTION OF THE DRAWINGS

Further objects, advantages and features of the present invention will be apparent from the description of an embodiment with reference to the attached drawings, in which:

FIG. 1 is a schematic side view of a locating apparatus according to the invention;

FIG. 2 is a front view of the locating apparatus according to the invention;

FIG. 3 illustrates schematically the shock wave therapy of a concrement;

FIG. 4 shows the projection d′ of a displacement distance d on the image plane;

FIG. 5 is a schematic representation to clarify the operation of the locating method according to the invention, and

FIG. 6 shows the process steps of the locating method according to the invention.

### DESCRIPTION OF A PREFERRED EMBODIMENT

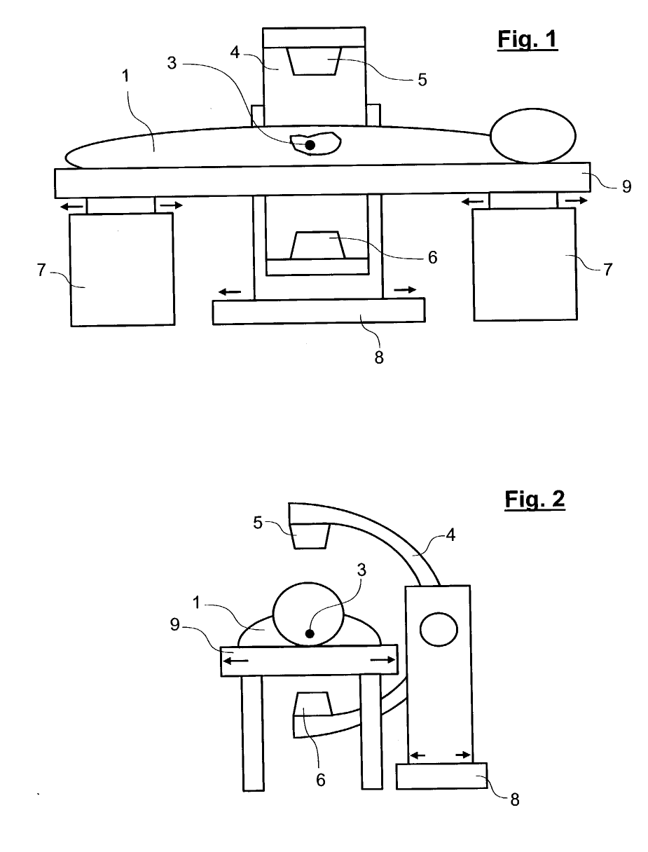

FIG. 1 shows a side view of an apparatus according to the invention for the three-dimensional location of a concrement 3 in the body of an organism, for example a patient 1. The patient lies on a treatment table 9 which is movable, for example, in the longitudinal and/or transverse direction of the body (see front view in FIG. 2). An X-ray appliance 4 (C-arc) as the imaging system is mounted on a pedestal 8 which permits movement of the X-ray appliance in the longitudinal and/or transverse direction of the patient. According to the invention it is sufficient if either the treatment table 9 or the X-ray appliance 4 is horizontally movable.

The imaging system 4 has a radiation source 5 which emits transilluminating X-radiation (AP projection) on to the body 1 and generates a projection image of the transilluminated body section in the image window 6. The distance z′ between radiation source 5 and image window or image plane 6 remains always constant during the location procedure. Moreover, only AP-projection images are produced.

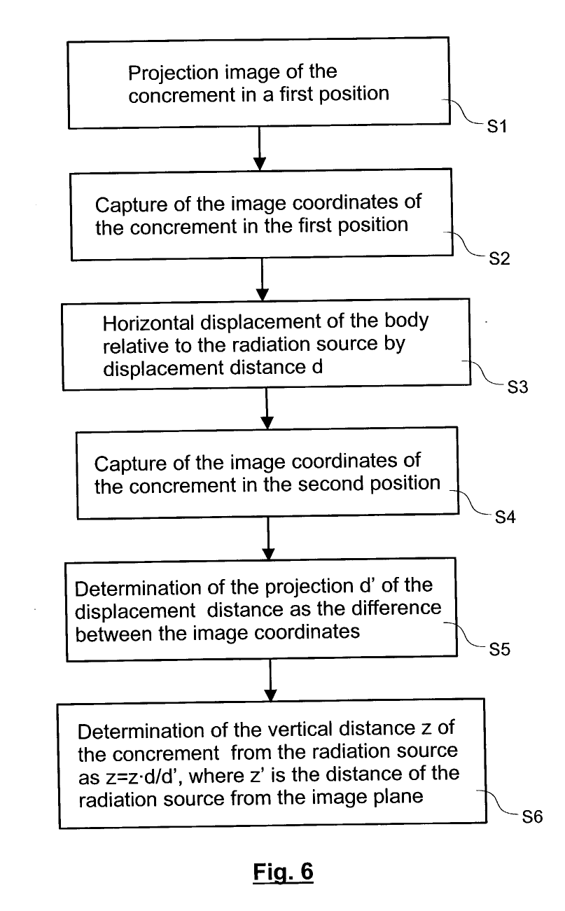

The locating method according to the invention is illustrated schematically in FIG. 6. The patient is first so positioned relative to the imaging device that the concrement 3 is located in a peripheral area of the image window of the imaging device, for example, in the right-hand top corner of the image field illustrated in FIG. 4. Then, in process step S1 (FIG. 6), a projection image of the concrement in this position is taken and the image coordinates of the concrement 3 are captured (step S2). The location of the concrement in the body plane (horizontal plane, X-Y plane) is therefore already achieved. In the next process step S3 for vertical location the body is displaced horizontally by the displacement distance d relative to the radiation source. This can be effected by moving the treatment table in the X and/or Y direction, or by moving the X-ray appliance in the X and/or Y direction. As this happens, the actual horizontal displacement distance d of the body relative to the imaging device is captured by means of a measuring device (not shown).

In steps S4 and S5 a projection image of the concrement 3 in the new position is generated and the projection d′ of the displacement distance as the difference between the image coordinates of the concrement in the two image positions is determined. In selecting the displacement distance d it should be ensured that the concrement remains within the image field of the imaging device. As the measuring accuracy increases with an increase in the displacement distance d, maximum utilisation of the image field, as shown in FIG. 4, is advantageous. In that Figure the projection d′ of the displacement distance from a starting position at top right to an end position at bottom left is shown.

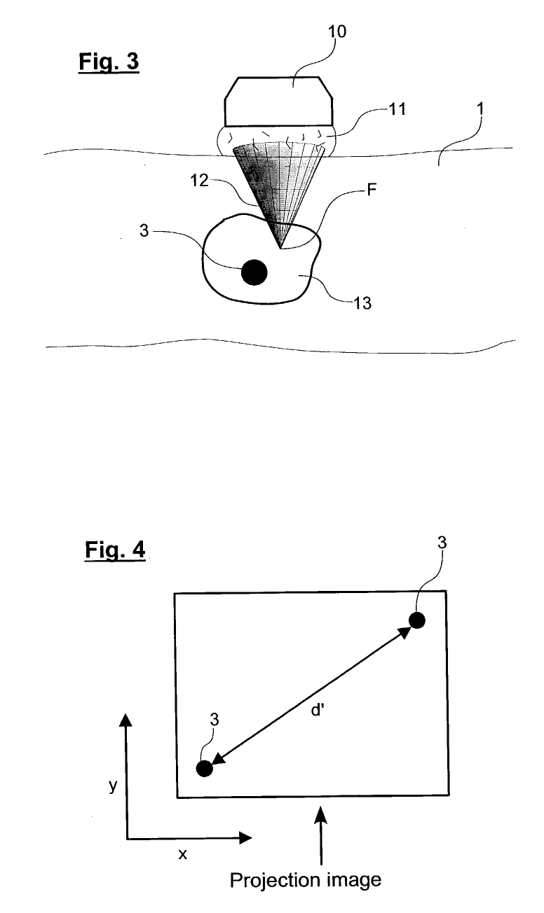

In the next process step S6 the vertical distance z of the concrement from the radiation source 5 is determined as

$$z = {z}^{′} \cdot \frac{d}{{d}^{′}} ,$$

where z′ is the distance of the radiation source 5 from the image plane 6. All three spatial coordinates X, Y and Z of the concrement are therefore known, so that the focal point F of the shock wave source 10 (see FIG. 3) can be made to coincide with the spatial position of the stone 3.

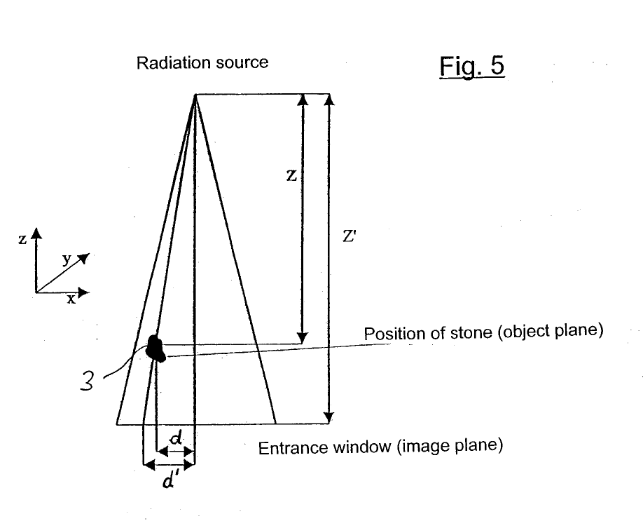

The determining of the vertical coordinate of the stone is described below with reference to FIG. 5. It follows from the set of rays that the vertical distance z of the stone from the radiation source, to the vertical distance z′ of the image plane from the radiation source, corresponds to the relationship of the actual displacement distance d to the projection d′ of the displacement distance. Consequently:

$$z = {z}^{′} \cdot \frac{d}{{d}^{′}} .$$

The distance from the radiation source and therefore the vertical position of the concrement can therefore be determined from the values: displacement distance d, projection of displacement distance d′ and distance between radiation source and image plane z′. The value z′ is constant and the values d and d′ can be simply determined by means of second vertical projection images. In this way, with the X and Y coordinates of the concrement, the spatial position (x, y, z) of the concrement to be treated in the body is obtained in a simple manner.

1. A method for the three-dimensional location of a concrement (3) in the body (1) of an organism for positioning a therapy device, comprising the steps:
generation of a vertical projection image of the concrement (3) by means of an imaging radiation source,
determination of the horizontal position of the concrement (3) by means of the projection image,
horizontal displacement of the body (1) relative to the radiation source (5) by a displacement distance d,
capture of the projection d′ of the displacement distance,
determination of the vertical position of the concrement (3) from the values: displacement distance d, projection d′ of the displacement distance and distance z′ of the radiation source (5) from the image plane (6) of the projection image.

2. A method according to claim 1, wherein the distance z of the concrement (3) from the position of the radiation source (5) is determined as
$$z = {z}^{′} \cdot \frac{d}{{d}^{′}} .$$

3. A method according to claim 1, wherein the radiation source (5) is an X-ray source.

4. A method according to claim 1, wherein the body (1) is moved by means of a movable treatment table (9) relative to the radiation source (5).

5. A method according to claim 1, wherein the radiation source (5) is rigidly connected to the image window of the projection device and is movable relative to the body (1).

6. A method according to claim 1, wherein the relative motion is effected in such a way that an available image field of the projection image is utilised to the maximum.

7. A method according to claim 6, wherein the displacement distance is disposed diagonally in a rectangular image field of the projection image.

8. An apparatus for locating a concrement in the body (1) of an organism for positioning a therapy device, comprising:
an imaging device (4) comprising a radiation source (5) and an image window (6) located at a fixed distance z′ from one another, for generating a vertical projection image of the concrement (3) and determinations of the horizontal position of the concrement,
an arrangement for horizontally displacing the body (1) relative to the radiation source (5) and for capturing the displacement distance d,
an arrangement for capturing the projection d′ of the displacement distance in the image window (6), and
an arrangement for determining the vertical position of the concrement from the values d, d′ and z′.

9. An apparatus according to claim 8, wherein the distance z of the concrement (3) from the radiation source (5) is determined as
$$z = {z}^{′} \cdot \frac{d}{{d}^{′}} .$$

10. An apparatus according to claim 8, wherein the radiation source is an X-ray source.

11. An apparatus according to claim 8, wherein the therapy device is a device for shock wave therapy.

12. An apparatus according to claims 8, comprising a treatment table (9) which is horizontally movable in the longitudinal and/or transverse direction of the body.

13. An apparatus according to claims 8, wherein the imaging device (4) is horizontally movable in the longitudinal and/or transverse direction of the body.
